(19) United States
(12) Patent Application Publication
(10) Pub. No.: US 2025/0031997 A1
(43) Pub. Date: Jan. 30, 2025

(54) FINGER TAPPING MEASUREMENT PROCESSING APPARATUS, METHOD, AND COMPUTER PROGRAM

(71) Applicant: Maxell, Ltd., Kyoto (JP)

(72) Inventors: Yoshiharu UCHIDA, Kyoto (JP); Ran TACHIBANA, Kyoto (JP); Tomohiko MIZUGUCHI, Kyoto (JP)

(73) Assignee: Maxell, Ltd., Kyoto (JP)

(21) Appl. No.: 18/715,753

(22) PCT Filed: Dec. 3, 2021
(86) PCT No.: PCT/JP2021/044465

§ 371 Date: Jun. 3, 2024

(51) Int. Cl. A61B 5/11; A61B 5/00; A61B 5/16; G01D 5/20; G06T 11/20

(52) U.S. Cl. CPC A61B 5/1101; A61B 5/165; A61B 5/4088; A61B 5/6826; A61B 5/7278; A61B 5/7425; G01D 5/20; G06T 11/206; A61B 2562/0223

(57) ABSTRACT

A finger tapping measurement processing apparatus, a method, and a computer program, which are capable of quantitatively evaluating a fatigue degree of fingers in a finger tapping motion, are provided. A finger tapping measurement processing apparatus in the present invention includes: a measurement detector including a tapping sensor that magnetically detects a finger tapping motion that is an opening and closing motion of two fingers; and a processor, which processes measurement data measured by the measurement detector. The processor includes: a feature amount extraction circuit, which extracts, as quantitative data, a feature amount related to the fatigue degree of the finger from detection information detected by the tapping sensor; and a time-series data generation circuit, which generates time-series data of the feature amount extracted by the feature amount extraction circuit.

### TECHNICAL FIELD

The present invention relates to a finger tapping measurement processing apparatus, a method, and a computer program for measuring a finger tapping motion and processing its measurement result.

### BACKGROUND ART

While the aging society is progressing, the number of patients with dementia of Alzheimer's type is increasing year by year. In a case where early detection is enabled, the progression of the disease can be delayed by medication. It is difficult to distinguish between symptoms associated with aging such as forgetfulness and the disease, and in many cases, after the disease becomes severe, a patient has a medical checkup at a hospital for the first time.

In such a situation, as screening inspections for early detection of the dementia of Alzheimer's type, a blood test, an olfactory test, a test in which medical inquiries by a doctor are reproduced on a tablet terminal, and the like have been conventionally performed. However, there is a problem that the subject bears a large burden, such as pain at the time of blood collection and the length of the inspection time. On the other hand, as an inspection with a small burden on the subject, cognitive function evaluation with button pressing or finger motion measurement of one hand with use of a tablet terminal is also carried out (see, for example, Patent Literature 1). However, there is a drawback that sufficient examination accuracy cannot be obtained. If it is possible to perform a simple screening inspection with high accuracy and with a small burden on the subject, such an inspection will lead to early detection of the dementia of Alzheimer's type, and can contribute to improvement in the quality of life of the patient and reductions of medical cost and care cost.

On the other hand, these years, it becomes clear that a motion pattern specific to the dementia of Alzheimer's type can be extracted from an opening and closing motion (a finger tapping motion) of two fingers that are the thumb and forefinger of each of both hands, and it is confirmed that there is a high correlation with dementia inspections of finger motion measurements and general medical inquiries. They are said that the finger tapping motion measurements show results of decrease in rhythm motion function of both fingers caused by shrinkage of brain in the dementia of Alzheimer's type. In addition, the fingers are said to be a second brain, and many regions in the brain are related to the functions of the fingers. The motions of the fingers are, without being limited to the dementia of Alzheimer's type, also said to be related to dementia such as cerebrovascular dementia and Lewy body dementia, Parkinson's disease, developmental coordination disorder (such as inability to skip or jump rope), and the like. That is, it becomes possible to know the state of the brain from the finger tapping motion. Furthermore, it is possible to quantify the dexterous motion function of fingers by utilizing the finger tapping motion of as a “measuring tool” indicating the health condition of the brain, and thus the finger tapping motion can be used in various fields such as a healthcare field, a rehabilitation field, and a life support field.

### CITATION LIST

### Patent Literature

Patent Literature 1: JP 2010-259634 A

### SUMMARY OF INVENTION

### Technical Problem

In the finger tapping motion that is an opening and closing motion of two fingers that are the thumb and the forefinger of a hand, a fatigue degree of the fingers during such a motion can be an important index for evaluating a progression degree of a disorder including dementia and a recovery degree of the motion function.

However, conventionally, in the finger tapping motion, there are some cases where inspectors such as doctors visually confirm and instinctively evaluate the number of times of the opening and closing motion of the fingers and an open situation of the fingers. In such cases, it is impossible to quantitatively evaluate the fatigue degree of the fingers.

The present invention has been made in view of the above circumstances, and it is an object of the present invention to provide a finger tapping measurement processing apparatus, a method, and a computer program capable of quantitatively evaluating a fatigue degree of fingers in a finger tapping motion.

### Solution to Problem

In order to solve the above problems, a finger tapping measurement processing apparatus according to the present invention includes: a measurement detector including a tapping sensor that magnetically detects a finger tapping motion that is an opening and closing motion of two fingers; and a processor that processes measurement data measured by the measurement detector, in which the processor includes: a feature amount extraction circuit that extracts, as quantitative data, a feature amount related to a fatigue degree of the fingers from detection information detected by the tapping sensor; and a time-series data generation circuit that generates time-series data of the feature amount extracted by the feature amount extraction circuit.

According to the above configuration of the present invention, the feature amount related to the fatigue degree of the fingers is extracted as the quantitative data from the detection information detected by the tapping sensor, and its time-series data is generated, so that the fatigue degree of the subject (a person to be subjected to measurement by the present apparatus, and the same will be apply, hereinafter) that changes over time can be quantitatively and clearly grasped. Therefore, it becomes possible to obtain an important index for evaluating the progression degree of a disorder including the dementia and the recovery degree of the motion function.

In addition, in the above configuration, the feature amount extracted by the feature amount extraction circuit preferably includes at least one of a phase difference between tapping waveforms of a right hand and a left hand in the finger tapping motion of cyclically opening and closing the fingers, a total motion distance accompanied by opening and closing the fingers, a tapping cycle in the finger tapping motion, and a maximum separated distance between the two fingers. These feature amounts are parameters directly indicating the fatigue degree over time in the finger tapping motion of the subject, thereby making it possible to directly and clearly grasp (evaluate) the fatigue degree in the finger tapping motion. In this case, as the fatigue degree in the finger tapping motion increases, it becomes difficult to open and close the fingers at a constant timing, and the variation increases in phase difference (shift in phase) between the tapping waveforms of the right hand and the left hand in the finger tapping motion of cyclically opening and closing the fingers. In addition, as the fatigue degree in the finger tapping motion increases, the opening and closing operation of the fingers slows down, and thus the tapping cycle (opening and closing time) in the finger tapping motion becomes longer, and the total motion distance accompanied by opening and closing the fingers also tends to decrease. Furthermore, as the fatigue degree in the finger tapping motion increases, the motion of the fingers also becomes smaller, and thus the maximum separated distance (maximum point) between the two fingers also decreases. In this manner, these parameters are directly related to the fatigue degree of the fingers (a direct index indicating the fatigue degree). Therefore, by quantitatively grasping them, it becomes possible to reliably grasp the progression degree of the disorder including the dementia and the recovery degree of the motion function. Note that the phase difference (shift in phase) in the finger tapping motion of cyclically opening and closing the fingers is obtained by, for example, extracting a shift in the tapping waveform of the left hand with respect to that of the right hand, in a case where one cycle of the tapping waveform of the right hand is set to 360 degrees.

Further, in the above configuration, the time-series data generation circuit preferably generates graphed time-series data. Such graphed time-series data enables quantitative evaluation at a glance.

In addition, in the above configuration, the finger tapping measurement processing apparatus preferably further includes a display that displays the time-series data generated by the time-series data generation circuit. In such a case, the processor preferably further includes an average value data generation circuit that generates average value data related to each feature amount of a plurality of subjects whose finger tapping motions are measured by the measurement detector, and the time-series data generation circuit generates display data to be displayed on the display in such a manner that a reference line indicating the average value data is superimposed on the time-series data. For such average value data, for example, by calculating the average value by age, it becomes possible to relatively evaluate whether the subject has a health condition appropriate to age, based on comparison with the actually measured value of the subject at present, and by displaying a reference line indicating such average value data in such a manner that the reference line is superimposed on the time-series data, it becomes possible to easily grasp the relative health condition of the subject at a glance.

Further, in the above configuration including the display, the time-series data generation circuit preferably generates display data to be displayed on the display in such a manner that past history data in the time-series data of an identical feature amount is arranged side by side. This makes it possible to grasp the progression degree of the disorder including the dementia and the recovery degree of the motion function at a glance.

In addition, in the above configuration including the display, the time-series data generation circuit preferably divides a time axis of the time-series data of the feature amount into a plurality of time zones each having an equal elapsed time, generates each section data that is the time-series data corresponding to each time zone, and also generates display data to be displayed on the display in such a manner that the each section data is arranged side by side along a continuous time series to be distinguishable from each other. This makes it possible to grasp the degree of gradual change in the fatigue degree in a series of time series at a glance as a temporal change in the inclination of the straight line.

Further, in the above configuration including the display, the time-series data generation circuit preferably divides a time axis of the time-series data of the feature amount into a plurality of time zones each having an equal elapsed time, generates each section data that is the time-series data corresponding to each time zone, and also generates display data to be displayed on the display in such a manner that the each section data is arranged side by side in each time series in each time zone to be distinguishable from each other. This makes it possible to grasp the degree of gradual change in the fatigue degree in a series of time series at a glance as an amount of difference in the inclination of the straight line.

Furthermore, in addition to the above configuration, the processor may evaluate, for example, the brain function and the cognitive function of the subject (for example, by comparison with data of a healthy person), based on the feature amount. Such evaluation is effective as screening at an early stage to determine dementia, and can be helpful in detecting the dementia. In addition, the application of the measurement processing apparatus including such a processor is not limited to a clinical field, and the scope of its application is wide. For example, the apparatus can also contribute to, determination of an ability to judge while driving a car, and is also applicable to a brain training game or the like.

Further, in addition to the above-described finger tapping measurement processing apparatus, the present invention also provides a finger tapping measurement processing method and a computer program for measuring a finger tapping motion and processing a measurement result.

### Advantageous Effects of Invention

According to the finger tapping measurement processing apparatus in the present invention, the feature amount related to the fatigue degree of the fingers is extracted as the quantitative data from detection information detected by the tapping sensor, and its time-series data is generated, so that the fatigue degree of the subject that changes over time can be quantitatively and clearly grasped.

### BRIEF DESCRIPTION OF DRAWINGS

FIG. 1 is a block diagram illustrating a schematic configuration of a finger tapping measurement processing apparatus according to an embodiment of the present invention.

FIG. 2 is a schematic view illustrating both hands of a subject in which tapping sensors are respectively attached to thumbs and forefingers.

FIG. 3 is a flowchart illustrating an example of an operation of the finger tapping measurement processing apparatus of FIG. 1.

FIG. 4 is a diagram illustrating an example of display data in which, with regard to left and right hands of one subject who performs finger tapping alternately, graphed time-series data of a feature amount, which is a maximum separated distance (maximum point) between two fingers, and graphed time-series data of a feature amount, which is a tapping cycle (opening and closing time) in a finger tapping motion, are displayed side by side, (a) illustrates display data of the right hand, and (b) illustrates display data of the left hand.

FIG. 5 is a diagram illustrating an example of graphed time-series data of a feature amount, which is a phase difference (simultaneous phase difference) between tapping waveforms of the right hand and the left hand in the finger tapping motion of cyclically opening and closing the right hand and the left hand simultaneously.

FIG. 6 is a diagram illustrating an example of graphed time-series data of a feature amount, which is a phase difference (alternate phase difference) between tapping waveforms of the right hand and the left hand in the finger tapping motion of cyclically opening and closing the right hand and the left hand alternately.

FIG. 7 is a diagram illustrating an example of display data in which a time axis of graphed time-series data of a feature amount, which is a maximum separated distance (maximum point) between two fingers, is divided into a plurality of time zones each having an equal elapsed time, and section data, which is time-series data corresponding to each time zone, is displayed side by side along a continuous time series.

FIG. 8 is a diagram illustrating an example of display data in which, with regard to the left and right hands of one subject who performs the finger tapping alternately, a time axis of graphed time-series data of a feature amount, which is a total motion distance accompanied by opening and closing the fingers, is divided into a plurality of time zones each having an equal elapsed time, and section data, which is time-series data corresponding to each time zone, are displayed along a continuous time series to be distinguishable from each other.

FIG. 9 is a diagram illustrating an example of display data in which, with regard to the left and right hands of one subject (the same subject with the case in FIG. 8) who performs the finger tapping alternately, a time axis of graphed time-series data of a feature amount, which is a total motion distance accompanied by opening and closing the fingers, is divided into a plurality of time zones each having an equal elapsed time, and section data, which is time-series data corresponding to each time zone, is displayed side by side in each time series in each time zone to be distinguishable from each other.

FIG. 10 is a diagram with regard to the left hand of another subject who performs the finger tapping alternately, (a) is a diagram illustrating an example of display data in which a time axis of graphed time-series data of a feature amount, which is a total motion distance accompanied by opening and closing the fingers, is divided into a plurality of time zones each having an equal elapsed time, and section data, which is time-series data corresponding to each time zone, is displayed along a continuous time series to be distinguishable from each other, and (b) is a diagram illustrating an example of display data in which the section data is displayed side by side in each time series in each time zone to be distinguishable from each other.

### DESCRIPTION OF EMBODIMENTS

Hereinafter, embodiments of the present invention will be described with reference to the drawings. In the present embodiment, the technologies to be described in the following are provided, and contribute to medical development and realization of health society with highly advanced technologies. The present measurement processing apparatus (method) is implemented, and contributes to the “Goal 9: Industry, Innovation, and Infrastructure” of the sustainable development goals (SDGs) proposed by the United Nations.

In addition, in the following embodiments, a finger tapping measurement processing apparatus and a method thereof will be described. However, the present invention may be configured as a computer program that enables a computer to perform measurement processing performed by the finger tapping measurement processing apparatus (method).

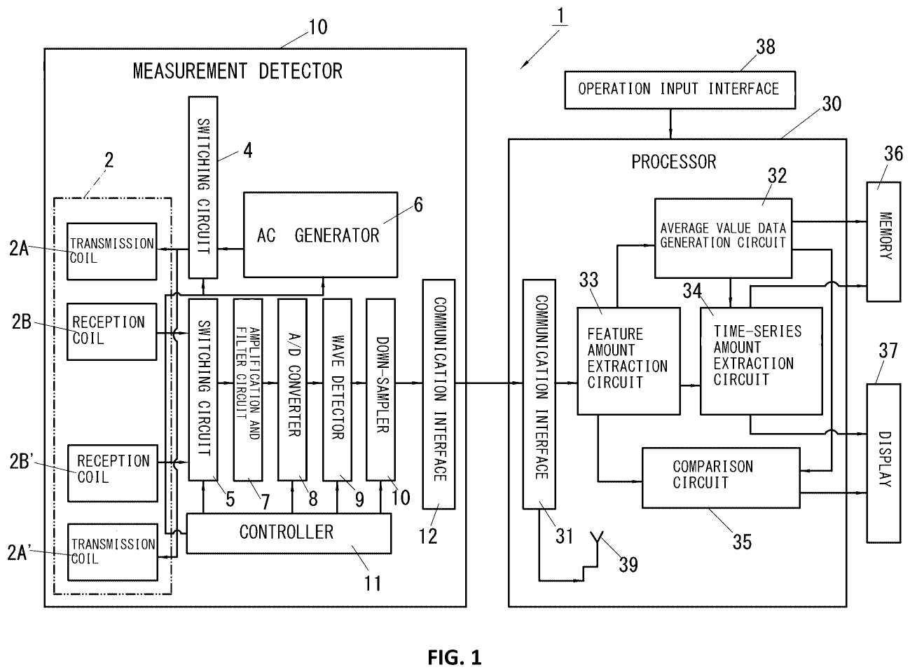

FIG. 1 illustrates a schematic configuration of a finger tapping measurement processing apparatus 1 according to an embodiment of the present invention. As illustrated in the drawing, a measurement detector 10 including a tapping sensor 2, which magnetically detects a finger tapping motion that is an opening and closing motion of two fingers, and a processor 30, which processes measurement data measured by the measurement detector 10, are included.

The measurement detector 10 calculates motion data of fingers based on a relative distance between a pair of a transmission coil and a reception coil that are attached to the fingers (or that may be attachable to other movable parts) of a living body. For example, the measurement detector 10 detects information of finger motions of a subject in a time-series manner, and is capable of acquiring motion information of the subject about at least any one of a distance, a speed, an acceleration, and a jerk degree (obtained by time-differentiating the acceleration), as time-series data (waveform data).

The measurement detector 10 includes a tapping sensor 2, first and second switching circuits 4 and 5, an AC generator 6 for generating an alternating current, an amplification and filter circuit 7, an A/D converter 8, a wave detector 9, a down-sampler 10, which performs down-sampling, and a controller 11, which controls operations of these components.

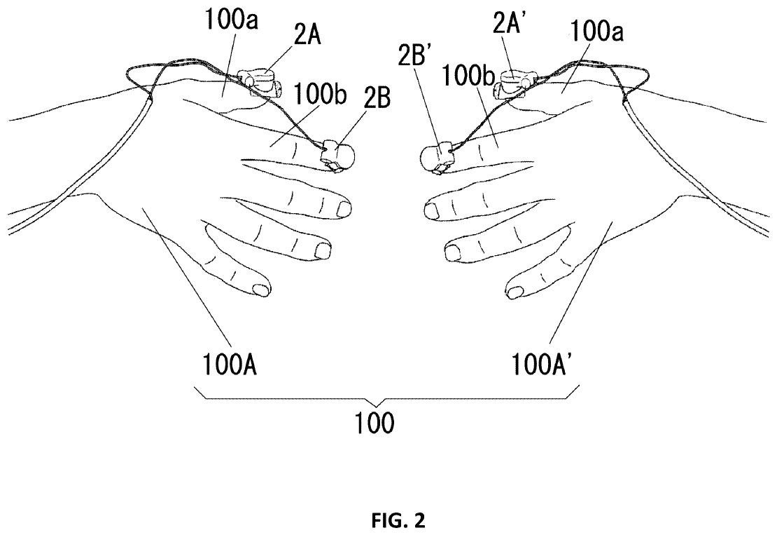

The tapping sensor 2 includes a pair of a transmission coil 2A (2A′) and a reception coil 2B (2B′) (which may be a plurality of coil pairs in a row), which are attached to fingers (for example, nail parts) of a hand 100 of the subject with, for example, a double-sided tape or a fixing band, as illustrated in FIG. 2. Specifically, in FIG. 2, a pair of the transmission coil 2A and the reception coil 2B are respectively attached to a thumb 100a and a forefinger 100b of a right hand 100A of the subject, and a pair of the transmission coil 2A′ and the reception coil 2B′ are respectively attached to the thumb 100a and the forefinger 100b of a left hand 100A′ of the subject (attached fingers may be reversed, or may be another finger). In this case, the transmission coil 2A (2A′) transmits a magnetic field, and the reception coil 2B (2B′) receives (detects) the magnetic field transmitted by the transmission coil 2A (2A′).

One AC generator 6 is connected with the transmission coil 2A (2A′) via the first switching circuit 4. A switching operation of the first switching circuit 4 causes an alternating current (for example, an electric current of 20 kHz) from the AC generator 6 to sequentially flow through the transmission coil 2A (2A′), and the transmission coil 2A (2A′) through which the alternating current has flown generates an AC magnetic field. The AC generator 6 generates an alternating current of a predetermined frequency, and the controller 11 controls a timing when the current flows. Note that a signal generated by the AC generator 6 is used as a reference signal in a detection operation of the wave detector 9.

The controller 11 generates a synchronization signal for controlling the first and second switching circuits 4 and 5. Such a synchronization signal enables the first switching circuit 4 and the second switching circuit 5 to be simultaneously switched, and each pair of the transmission coil 2A (2A′) and the reception coil 2B (2B′) sequentially operate.

In addition, the reception coil 2B (2B′) is connected with the amplification and filter circuit 7 via the second switching circuit 5, an output signal from the amplification and filter circuit 7 is converted into a digital signal by the A/D converter 8, and the digital signal is transmitted to the wave detector 9. Note that the A/D converter 8 converts analog data into digital data, and thus facilitates subsequent processing (down-sampling or the like). Further, out of the AC magnetic field waveforms detected by the reception coil 2B (2B′), the wave detector 9 also performs processing of deleting an AC magnetic field waveform (noise part) for a predetermined period immediately after the second switching circuit 5 switches.

In addition, the controller 11 accurately controls the time of deletion processing in the AC magnetic field waveform of each of the reception coils 2B (2B′). After such deletion processing, the wave detector 9 performs full-wave rectification processing and filter processing (mainly processing on a low-pass filter (LPF)) with use of the above-described reference signal. Finally, the digital signal that has been processed by the wave detector 9 is converted (down-sampled) by the down-sampler 10 into coarse data of a sampling frequency (for example, 200 Hz) that is approximately 1/1000 (a predetermined ratio of) the sampling frequency (for example, 200 kHz) in the A/D converter 8. This enables reduction of the entire data capacity. Therefore, an output signal can be transmitted at a high speed, as data of a plurality of reception coils, even though the communication capacity is limited. That is, the data amount received from the down-sampler 10 is small, and thus a communication interface 12 of the measurement detector 10 is capable of delivering the motion data of the fingers related to the plurality of reception coils to the processor 30 (through a communication interface 31 of the processor 30) at a time in a wireless or wired manner.

The processor 30 processes measurement data that has been measured by the measurement detector 10. The processor 30 includes: a feature amount extraction circuit 33, which extracts, as quantitative data, a feature amount related to the fatigue degree of fingers from detection information detected by the tapping sensor 2 (that is, output data output from the measurement detector 10); a time-series data generation circuit 34, which generates time-series data of the feature amount extracted by the feature amount extraction circuit 33; an average value data generation circuit 32, which receives the feature amount from the feature amount extraction circuit 33, and which also generates (performs an arithmetic operation for) average value data (for example, calculates an average value a subject by age) related to each feature amount of a plurality of subjects whose finger tapping motions are measured by the measurement detector 10; and a comparison circuit 35, which compares actual measurement value data of the feature amount received from the feature amount extraction circuit 33 with the average value data received from the average value data generation circuit 32, and which outputs a comparison result. In particular, in the present embodiment, the time-series data generation circuit 34 generates graphed time-series data of the feature amount. Note that, as will be described later, the feature amount extracted by the feature amount extraction circuit 33 includes at least one of a phase difference (shift in phase) between the tapping waveforms of the right hand and the left hand in the finger tapping motion of cyclically opening and closing the fingers, a total motion distance accompanied by opening and closing the fingers, a tapping cycle (opening and closing time) in the finger tapping motion, and a maximum separated distance (maximum point) between the two fingers.

In addition, the finger tapping measurement processing apparatus 1 further includes: a display 37, which displays the time-series data generated by the time-series data generation circuit 34 of the processor 30 and the comparison result output from the comparison circuit 35 of the processor 30; a memory 36, which stores various data including the time-series data generated by the time-series data generation circuit 34 of the processor 30 and the average value data generated by the average value data generation circuit 32 of the processor 30; and an operation input interface 38, into which necessary data and an instruction to the processor 30 can be input by an operation.

In the above configuration, the processor 30 includes a CPU and the like, and executes programs such as an operating system (OS) and various operation control applications stored in the memory 36 to perform operation control processing of the above-described various circuits 32, 33, 34, and 35 and also to control startup operations of the various applications.

The memory 36 includes a flash memory or the like, and stores programs such as an operating system and an operation control application for various types of processing for image, audio, document, display, measurement, and the like. In addition, the memory 36 stores base data necessary for basic operations on the operating system or the like and information data such as file data used in various applications or the like.

Note that the processing in the processor 30 may be stored as one application, and measurement processing of the finger motions and calculation analyses of various feature amounts may be carried out in accordance with startup of the application. In addition, an external server apparatus or the like with high arithmetic processing performance and with a large capacity may receive a measurement result that has been measured from an information processing terminal, and may calculate and analyze the feature amount.

In addition, input means, such as a keyboard, a key button, a touch key, or the like, is generally used for the operation input interface 38. However, for example, a gesture operation or a voice input may be used, and the subject sets and inputs information that should be input.

Further, the communication interface 31 may not only receive the measurement result from the measurement detector 10 but also perform wireless communication with a server apparatus or the like in another place on short-distance wireless communication, wireless LAN, or base station communication. In such a case, on wireless communication, the measurement data, the feature amount that has been analyzed and calculated, and the like may be transmitted to and received from a server apparatus or the like through a transmission and reception antenna 39. Note that on short-range wireless communication, for example, an electronic tag is used. However, without being limited to this, any type of wireless LAN such as Bluetooth (registered trademark), infrared data association (IrDA, registered trademark), Zigbee (registered trademark), home radio frequency (HomeRF, Registered Trademark), or Wi-Fi (registered trademark) may be used, as long as it is capable of communicating wirelessly, when it is located near another information terminal. In addition, on base station communication, it is sufficient to use wireless communication over a long distance such as wideband code division multiple access (W-CDMA) or global system for mobile communications (GSM) (registered trademark). Note that it is also possible to detect a positional relationship or a direction between terminals with use of an ultra wide band (UWB). Although not illustrated, the communication interface 31 may use another method such as communication using optical communication sound waves as means for the wireless communication. In such a case, instead of the transmission and reception antenna 39, a light emitting and receiving unit and a sound wave output and sound wave input interface are each used.

Note that in the present embodiment, the measurement detector 10 and the processor 30 individually includes the above-described respective component elements, but may include a functional unit that integrates at least some or all of these component elements. The point is that any configuration form may be made, as long as the function of each of these component elements is ensured.

Further, in the present embodiment, the above-described time-series data generation circuit 34 of the processor 30 also has a function of generating various display data for displaying the generated time-series data in various display modes on the display 37. Specifically, the time-series data generation circuit 34 is capable of generating display data to be displayed on the display 37 in such a manner that a reference line indicating the average value data generated by the average value data generation circuit 32 is superimposed on the time-series data, and is also capable of generating display data to be displayed on the display 37 in such a manner that history data in the past in the time-series data of the same feature amount is arranged side by side. In addition, the time-series data generation circuit 34 is capable of dividing the time axis of the time-series data of the feature amount into a plurality of time zones each having an equal elapsed time to generate section data that is time-series data corresponding to each time zone, and is also capable of generating display data to be displayed on the display 37 in such a manner that each section data is arranged side by side along a continuous time series to be distinguishable from each other, or is also capable of generating display data to be displayed on the display 37 in such a manner that each section data is arranged side by side in each time series in each time zone to be distinguishable from each other.

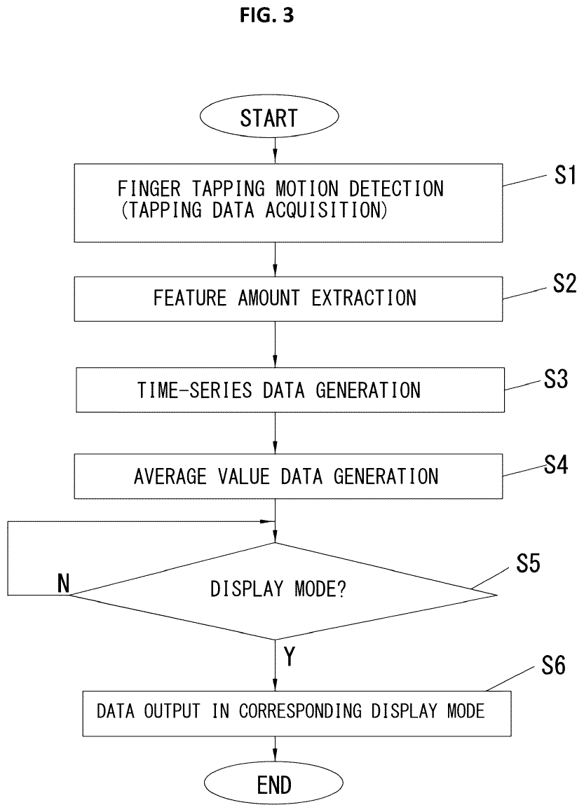

Next, an example of the operation of the finger tapping measurement processing apparatus 1 having the above-described configuration will be described in more detail with reference to the flowchart of FIG. 3 and FIGS. 4 to 10, including the display mode based on these display data.

FIG. 3 illustrates an example of processing steps performed by the processor 30. As illustrated in the drawing, in the finger tapping measurement processing apparatus 1 in the present embodiment, first, a finger tapping motion performed by a subject is detected (step S1). In this case, the measurement detector 10 magnetically detects the finger tapping motion of the subject with use of the tapping sensor 2 (a detection step), and the processor 30 acquires detection data from the tapping sensor 2 (a tapping data acquisition step). The finger tapping motion of the subject is detected by the measurement detector 10, and the detection information is received by the processor 30 in this manner. Subsequently, the processor 30 causes the feature amount extraction circuit 33 to extract a feature amount related to the fatigue degree of the fingers as quantitative data from the detection information (step S2: a feature amount extraction step), and in addition, causes the time-series data generation circuit 34 to generate time-series data (in the present embodiment, in particular, graphed time-series data) of the extracted feature amount (step S3: a time-series data generation step). In addition, in parallel to the above steps or after the above steps, the processor 30 causes the average value data generation circuit 32 to generate average value data related to each feature amount of the subject (step S4: an average value data generation step).

Then, for example, when a display mode is selected (or instructed) via the operation input interface 38 (step S5), the display data (time-series data) in the corresponding display mode that has been selected (instructed) is output from the time-series data generation circuit 34, and is displayed on the display 37 (step S6: a display step).

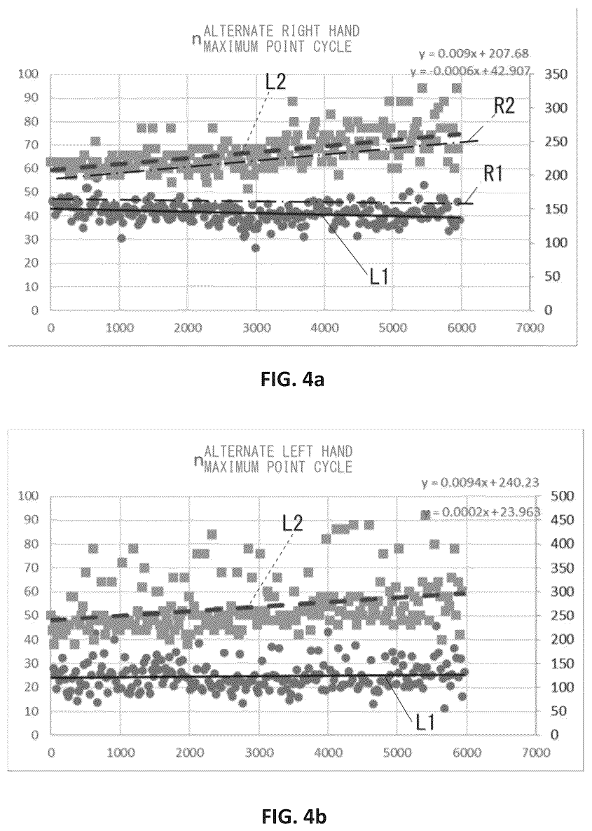

FIG. 4 illustrates an example of a display mode (display data) of displaying time-series data of the feature amount, which is the maximum separated distance (maximum point) between the two fingers, and time-series data of a feature amount, which is the tapping cycle (opening and closing time) in the finger tapping motion, side by side. Specifically, on a lower side in (a) of FIG. 4, with regard to the right hand of one subject “n” who performs the finger tapping motion with the right hand and the left hand alternately, time-series data of the maximum separated distance (maximum point) between the two fingers is illustrated as a scatter diagram with circle dots, and a solid straight line (approximate straight line) L1 (y=−0.0006x+42.907) indicating substantially the average values of them, that is, graphed time-series data is also illustrated. In this case, the horizontal axis represents time (×10 ms), and the vertical axis (vertical axis on the left side) represents distance (mm). On the other hand, on an upper side in (a) of FIG. 4, with regard to the right hand of the same subject “n” who performs the finger tapping motion with the right hand and the left hand alternately, time-series data of the tapping cycle (opening and closing time) is illustrated as a scatter diagram with square dots, and a dashed straight line (approximate straight line) L2 (y=0.009x+207.68) indicating substantially the average values of them, that is, graphed time-series data is also illustrated. In this case, the horizontal axis represents time (×10 ms), and the vertical axis (vertical axis on the right side) represents tapping cycle (ms). On the other hand, on a lower side in (b) of FIG. 4, with regard to the left hand of the same subject “n” who performs the finger tapping motion with the right hand and the left hand alternately, time-series data of the maximum separated distance (maximum point) between the two fingers is illustrated as a scatter diagram with circle dots, and a solid straight line (approximate straight line) L1 (y=0.0002x+23.963) indicating substantially the average values of them, that is, graphed time-series data is also illustrated. Also in this case, the horizontal axis represents time (×10 ms), and the vertical axis (vertical axis on the left side) represents distance (mm). On the other hand, on an upper side in (b) of FIG. 4, with regard to the left hand of the same subject “n” who performs the finger tapping motion with the right hand and the left hand alternately, time-series data of the tapping cycle (opening and closing time) is illustrated as a scatter diagram with square dots, and a dashed straight line (approximate straight line) L2 (y=0.0094x+240.23) indicating substantially the average values of them, that is, graphed time-series data is also illustrated. Also in this case, the horizontal axis represents time (×10 ms), and the vertical axis (vertical axis on the right side) represents tapping cycle (ms). As can be understood from these display data, as the fatigue degree in the finger tapping motion increases, the motions of the fingers also become smaller, and thus the maximum separated distance (maximum point) between the two fingers also decreases. In addition, as the fatigue degree in the finger tapping motion increases, the opening and closing operation of the fingers slows down, and thus the tapping cycle (opening and closing time) in the finger tapping motion becomes longer. Note that here, the graphed time-series data of the feature amount, which is the maximum separated distance (maximum point) between the two fingers, and the graphed time-series data of the feature amount, which is the tapping cycle (opening and closing time) in the finger tapping motion, are distinguished by the shape of the dot or the type of the straight line, but may be distinguished by another identification form such as a difference in color.

In addition, in such a display mode (similarly to the other display modes to be described below), for example, in accordance with selection (instruction) from the operation input interface 38, the time-series data generation circuit 34 may display as the display data on the display 37 in such a manner that reference lines indicating the average value data generated by the average value data generation circuit 32 (for example, a reference line R1 related to the maximum separated distance (maximum point) between the two fingers and a reference line R2 related to the tapping cycle (opening and closing time)) are superimposed on the time-series data (the straight lines L1 and L2 and their corresponding dots). The reference lines R1 and R2 based on such average value data (for example, the average value of all the measured subjects by age) make it possible to relatively evaluate whether the subject has a health condition appropriate to age, based on comparison with the actually measured values of the subject at present, and to easily grasp a relative health condition of the subject at a glance. In addition to or instead of displaying such reference lines R1 and R2, the comparison result from the comparison circuit 35, which compares the actually measured value data of the feature amount received from the feature amount extraction circuit 33 with the average value data received from the average value data generation circuit 32, may be displayed on the display 37 in the form of, for example, text data or the like.

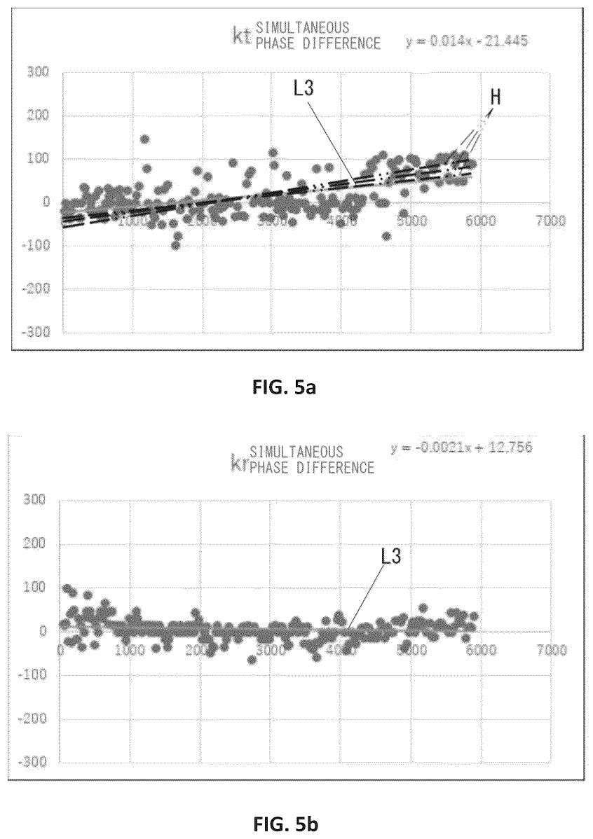

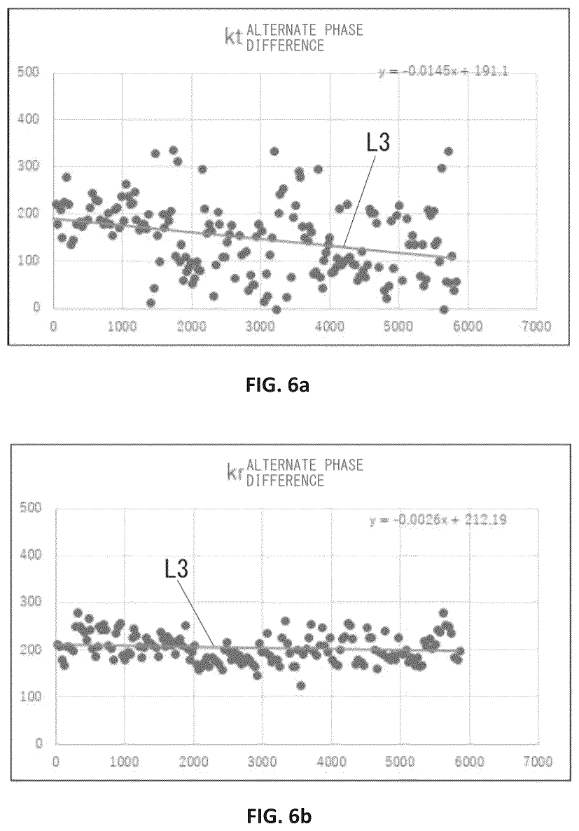

FIG. 5 illustrates an example of a display mode (display data) of displaying time-series data of a feature amount, which is a phase difference (simultaneous phase difference) between tapping waveforms of the right hand and the left hand in the finger tapping motion of cyclically opening and closing the right hand and the left hand simultaneously. Specifically, in (a) of FIG. 5, with regard to one subject “kt” who performs the finger tapping motion with the right hand and the left hand simultaneously, time-series data of the phase difference between the tapping waveforms of the right hand and the left hand in the finger tapping motion of cyclically opening and closing the fingers is illustrated as a scatter diagram with circle dots, and a solid straight line (approximate straight line) L3 (y=0.014x−21.445) indicating substantially the average values of them, that is, graphed time-series data is also illustrated. In this case, the horizontal axis represents time (×10 ms), and the vertical axis represents phase difference (°). On the other hand, in (b) of FIG. 5, with regard to another subject “kr” who performs the finger tapping motion with the right hand and the left hand simultaneously, time-series data of the phase difference between the tapping waveforms of the right hand and the left hand in the finger tapping motion of cyclically opening and closing the fingers is illustrated as a scatter diagram with circle dots, and a solid straight line (approximate straight line) L3 (y=−0.0021x+12.756) indicating substantially the average values of them, that is, graphed time-series data is also illustrated. Also in this case, the horizontal axis represents time (×10 ms), and the vertical axis represents phase difference (°). Further, FIG. 6 illustrates an example of a display mode (display data) of displaying time-series data of a feature amount, which is a phase difference (alternate phase difference) between tapping waveforms of the right hand and the left hand in the finger tapping motion of cyclically opening and closing the right hand and the left hand alternately. Specifically, in (a) of FIG. 6, with regard to the same subject “kt” with the case in (a) of FIG. 5, time-series data of the phase difference between the tapping waveforms of the right hand and the left hand in the finger tapping motion of cyclically opening and closing the fingers is illustrated as a scatter diagram with circle dots, and a solid straight line (approximate straight line) L3 (y=−0.0145x+191.1) indicating substantially the average values of them, that is, graphed time-series data is also illustrated. In this case, the horizontal axis represents time (×10 ms), and the vertical axis represents phase difference (°). On the other hand, in (b) of FIG. 6, with regard to the same subject “kr” with the case in (b) of FIG. 5, time-series data of the phase difference between the tapping waveforms of the right hand and the left hand in the finger tapping motion of cyclically opening and closing the fingers is illustrated as a scatter diagram with circle dots, and a solid straight line (approximate straight line) L3 (y=−0.0026x+212.19) indicating substantially the average values of them, that is, graphed time-series data is also illustrated. Also in this case, the horizontal axis represents time (×10 ms), and the vertical axis represents phase difference (°).

As can be understood from these display data, depending on the recovery degree from the disorder, as illustrated in (a) of FIG. 5 and (a) of FIG. 6, as the fatigue degree in the finger tapping motion increases, it becomes difficult to open and close the fingers at a constant timing. This increases variations in the phase difference (shift in phase) between the tapping waveforms of the right hand and the left hand in the finger tapping motion of cyclically opening and closing the fingers. However, as illustrated in (b) of FIG. 5 and (b) of FIG. 6, healthy persons have small variations in the phase difference. In the finger tapping motion of cyclically opening and closing the right hand and the left hand simultaneously, the phase difference is maintained at substantially 0 degrees. In the finger tapping motion of cyclically opening and closing the right hand and the left hand alternately, the phase difference is maintained at substantially 180 degrees.

In addition, in such a display mode (in a similar manner to the other display modes to be described below or that have been described above), for example, the selection (instruction) from the operation input interface 38 may cause the time-series data generation circuit 34 to display past history data H (here, a plurality of graphed history data in the past like a liner shape) in the time-series data of the same feature amount (here, the time-series data of the phase difference) side by side as the display data on the display 37, for example, as illustrated in (a) of FIG. 5.

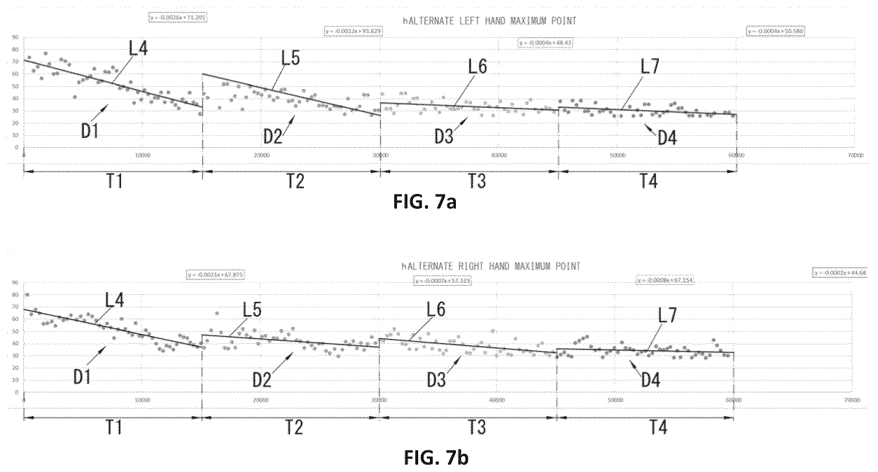

FIG. 7 illustrates another example of the display mode (display data) of displaying time-series data of a feature amount, which is the maximum separated distance (maximum point) between the two fingers (the horizontal axis represents time (×10 ms) and the vertical axis represents distance (mm)). Here, the time axis of the time-series data is divided into a plurality of time zones each having an equal elapsed time. Specifically, the measurement time of 60 seconds as a whole is divided into four time zones T1, T2, T3, and T4 at intervals of 15 seconds, and section data D1, D2, D3, and D4, which are time-series data corresponding to the respective time zones T1, T2, T3, and T4, are displayed side by side along a continuous time series. More specifically, in (a) of FIG. 7, with regard to the left hand of one subject “h” who performs the finger tapping motion with the right hand and the left hand alternately, in the time zone T1 from 0 seconds to 15 seconds, as the section data D1, time-series data of the maximum separated distance (maximum point) between the two fingers is illustrated as a scatter diagram with circle dots, and a solid straight line (approximate straight line) L4 (y=−0.0026x+71.201) indicating substantially the average values of them, that is, graphed time-series data is also illustrated. In addition, in the time zone T2 from 16 seconds to 30 seconds, as the section data D2, time-series data of the maximum separated distance (maximum point) between the two fingers is illustrated as a scatter diagram with circle dots, and a solid straight line (approximate straight line) L5 (y=−0.0022x+93.629) indicating substantially the average values of them, that is, graphed time-series data is also illustrated. Further, in the time zone T3 from 31 seconds to 45 seconds, as the section data D3, time-series data of the maximum separated distance (maximum point) between the two fingers is illustrated as a scatter diagram with circle dots, and a solid straight line (approximate straight line) L6 (y=−0.0004x+48.43) indicating substantially the average values of them, that is, graphed time-series data is also illustrated. Furthermore, in the time zone T4 from 46 seconds to 60 seconds, as the section data D4, time-series data of the maximum separated distance (maximum point) between the two fingers is illustrated as a scatter diagram with circle dots, and a solid straight line (approximate straight line) L7 (y=−0.0004x+50.586) indicating substantially the average values of them, that is, graphed time-series data is also illustrated.

In (b) of FIG. 7, with regard to the right hand of the same subject “h” who performs the finger tapping motion with the right hand and the left hand alternately, in the time zone T1 from 0 seconds to 15 seconds, as the section data D1, time-series data of the maximum separated distance (maximum point) between the two fingers is illustrated as a scatter diagram with circle dots, and a solid straight line (approximate straight line) L4 (y=−0.0021x+67.875) indicating substantially the average values of them, that is, graphed time-series data is also illustrated. In addition, in the time zone T2 from 16 seconds to 30 seconds, as the section data D2, time-series data of the maximum separated distance (maximum point) between the two fingers is illustrated as a scatter diagram with circle dots, and a solid straight line (approximate straight line) L5 (y=−0.0007x+57.319) indicating substantially the average values of them, that is, graphed time-series data is also illustrated. Further, in the time zone T3 from 31 seconds to 45 seconds, as the section data D3, time-series data of the maximum separated distance (maximum point) between the two fingers is illustrated as a scatter diagram with circle dots, and a solid straight line (approximate straight line) L6 (y=−0.0008x+67.154) indicating substantially the average values of them, that is, graphed time-series data is also illustrated. Furthermore, in the time zone T4 from 46 seconds to 60 seconds, as the section data D4, time-series data of the maximum separated distance (maximum point) between the two fingers is illustrated as a scatter diagram with circle dots, and a solid straight line (approximate straight line) L7 (y=−0.0002x+44.68) indicating substantially the average values of them, that is, graphed time-series data is also illustrated.

As can be understood from these display data, as the fatigue degree in the finger tapping motion increases, the motions of the fingers also become smaller, and thus the maximum separated distance (maximum point) between the two fingers also decreases in a later time zone, and the inclinations of the straight lines L4 to L7 gradually decrease. Note that in this display mode, the respective time zones may be displayed in a distinguishable manner, by changing a line type of the straight line or colors of the dots.

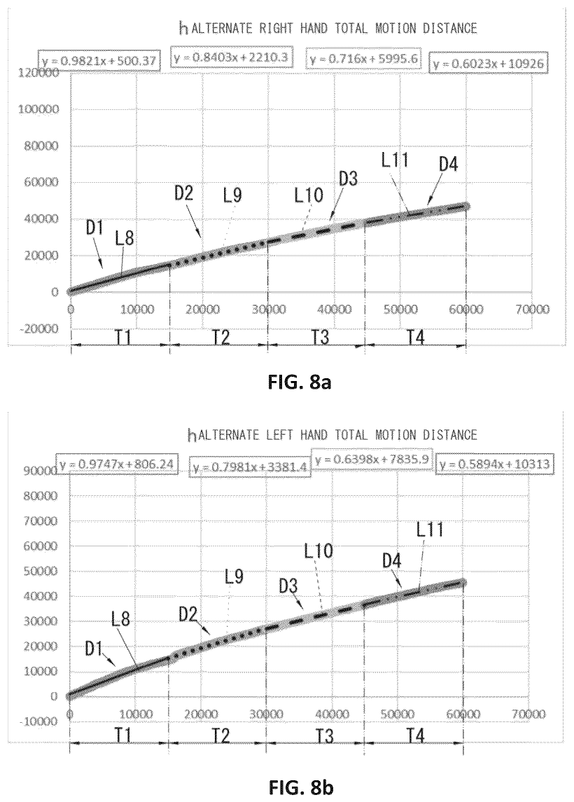

FIG. 8 illustrates an example of a display mode (display data) of displaying the time-series data of a feature amount, which is the total motion distance accompanied by opening and closing the fingers (the horizontal axis represents time (×10 ms) and the vertical axis represents distance (mm)). Here, the time axis of the time-series data is divided into a plurality of time zones each having an equal elapsed time. Specifically, the measurement time of 60 seconds as a whole is divided into four time zones T1, T2, T3, and T4 at intervals of 15 seconds, and section data D1, D2, D3, and D4, which are time-series data corresponding to the respective time zones T1, T2, T3, and T4, are displayed side by side along a continuous time series to be distinguishable from one another. More specifically, in (a) of FIG. 8, with regard to the right hand of one subject “h” who performs the finger tapping motion with the right hand and the left hand alternately, in the time zone T1 from 0 seconds to 15 seconds, as the section data D1, time-series data of the total motion distance accompanied by opening and closing the fingers is illustrated as a scatter diagram with circle dots, and a solid straight line (approximate straight line) L8 (y=0.9821x+500.37) indicating substantially the average values of them, that is, graphed time-series data is also illustrated. In addition, in the time zone T2 from 16 seconds to 30 seconds, as the section data D2, time-series data of the total motion distance accompanied by opening and closing the fingers is illustrated as a scatter diagram with circle dots, and a solid dotted line (approximate straight line) L9 (y=0.8403+2210.3) indicating substantially the average values of them, that is, graphed time-series data is also illustrated. Further, in the time zone T3 from 31 seconds to 45 seconds, as the section data D3, time-series data of the total motion distance accompanied by opening and closing the fingers is illustrated as a scatter diagram with circle dots, and a dashed straight line (approximate straight line) L10 (y=0.716x+5995.6) indicating substantially the average values of them, that is, graphed time-series data is also illustrated. Furthermore, in the time zone T4 from 46 seconds to 60 seconds, as the section data D4, time-series data of the total motion distance accompanied by opening and closing the fingers is illustrated as a scatter diagram with circle dots, and a two-dot chain straight line (approximate straight line) L11 (y=0.6023x+10926) indicating substantially the average values of them, that is, graphed time-series data is also illustrated.

In (b) of FIG. 8, with regard to the left hand of the same subject “h” who performs the finger tapping motion with the right hand and the left hand alternately, in the time zone T1 from 0 seconds to 15 seconds, as the section data D1, time-series data of the total motion distance accompanied by opening and closing the fingers is illustrated as a scatter diagram with circle dots, and a solid straight line (approximate straight line) L8 (y=0.9747x+806.24) indicating substantially the average values of them, that is, graphed time-series data is also illustrated. In addition, in the time zone T2 from 16 seconds to 30 seconds, as the section data D2, time-series data of the total motion distance accompanied by opening and closing the fingers is illustrated as a scatter diagram with circle dots, and a dotted straight line (approximate straight line) L9 (y=0.7981+3381.4) indicating substantially the average values of them, that is, graphed time-series data is also illustrated. Further, in the time zone T3 from 31 seconds to 45 seconds, as the section data D3, time-series data of the total motion distance accompanied by opening and closing the fingers is illustrated as a scatter diagram with circle dots, and a dashed straight line (approximate straight line) L10 (y=0.6398x+7835.9) indicating substantially the average values of them, that is, graphed time-series data is also illustrated. Furthermore, in the time zone T4 from 46 seconds to 60 seconds, as the section data D4, time-series data of the total motion distance accompanied by opening and closing the fingers is illustrated as a scatter diagram with circle dots, and a two-dot chain straight line (approximate straight line) L11 (y=0.5894x+10313) indicating substantially the average values of them, that is, graphed time-series data is also illustrated.

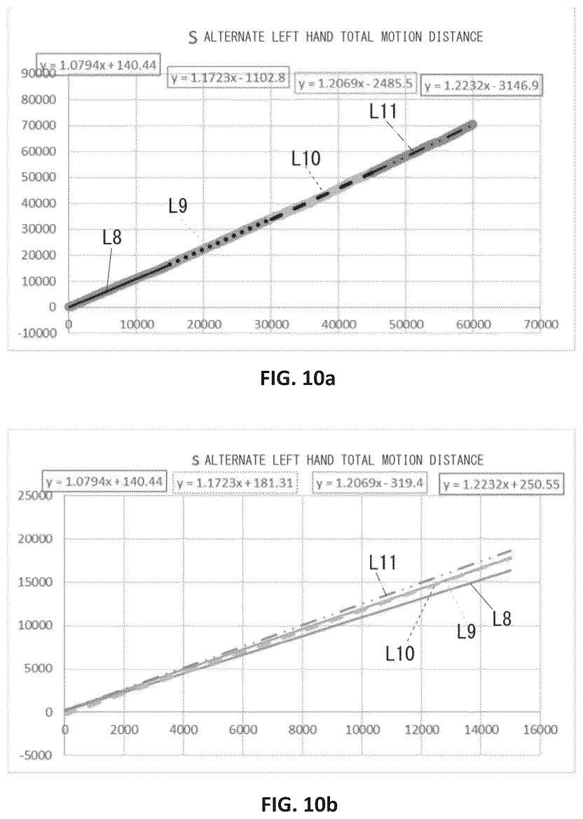

As can be understood from these display data, as the fatigue degree in the finger tapping motion increases, the opening and closing operation of the fingers slows down, and thus the total motion distance accompanied by opening and closing the fingers also tends to decrease in a later time zone, and the inclination of the straight line also decreases. However, in the case of a healthy person “s”, as illustrated in (a) of FIG. 10, the inclinations of the straight lines L8 (y=1.0794x+140.44), L9 (y=1.1723x+1102.8), L10 (y=1.2069x+2485.5), and L11 (y=1.2232x+3146.9) are maintained substantially constant regardless of the time zone. Note that in this display mode, each time zone may be displayed in a distinguishable manner by changing not only the line type of the straight line but also its color or the like.

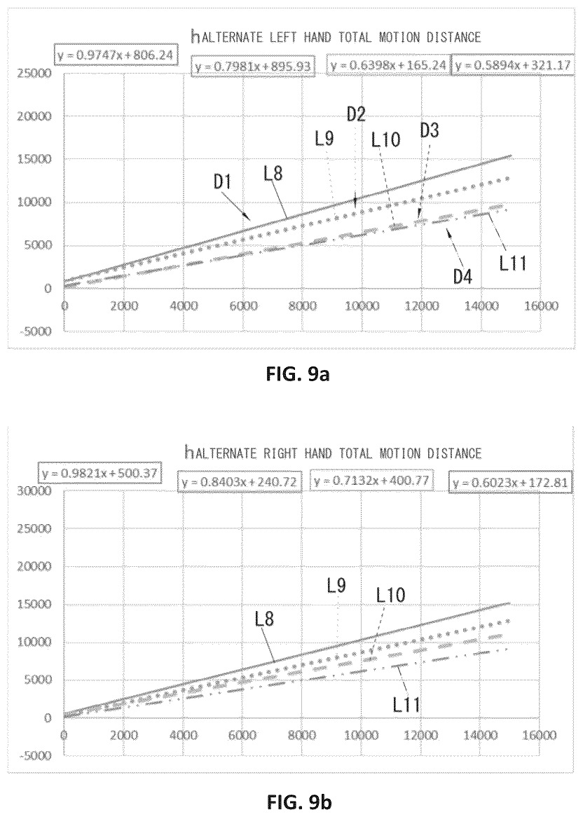

FIG. 9 illustrates another example of the display mode (display data) of displaying time-series data of a feature amount, which is the total motion distance accompanied by opening and closing the fingers (the horizontal axis represents time (×10 ms) and the vertical axis represents distance (mm)). Here, the time axis of the time-series data is divided into a plurality of time zones each having an equal elapsed time. Specifically, the measurement time of 60 seconds as a whole is divided into four time zones at intervals of 15 seconds, and section data D1, D2, D3, and D4, each of which is time-series data corresponding to each time zone, is displayed side by side in each time series in each time zone to be distinguishable from one another (with origins of the respective time series aligned). More specifically, in (a) of FIG. 9, with regard to the left hand of the same subject “h” of FIG. 8 who performs the finger tapping motion with the right hand and the left hand alternately, within a time section of 15 seconds, the solid straight line (approximate straight line) L8 in (b) of FIG. 8 as the section data D1, the dotted straight line (approximate straight line) L9 in (b) of FIG. 8 as the section data D2, the dashed straight line (approximate straight line) L10 in (b) of FIG. 8 as the section data D3, and the two-dot chain straight line (approximate straight line) L11 in (b) of FIG. 8 as the section data D4 are illustrated side by side with the origins of them aligned. In addition, in (b) of FIG. 9, with regard to the right hand of the same subject “h” of FIG. 8 who performs the finger tapping motion with the right hand and the left hand alternately, within a time section of 15 seconds, the solid straight line (approximate straight line) L8 in (a) of FIG. 8 as the section data D1, the dotted straight line (approximate straight line) L9 in (a) of FIG. 8 as the section data D2, the dashed straight line (approximate straight line) L10 in (a) of FIG. 8 as the section data D3, and the two-dot chain straight line (approximate straight line) L11 in (a) of FIG. 8 as the section data D4 are illustrated side by side with the origins of them aligned.

As can be understood from these display data, as the fatigue degree in the finger tapping motion increases, the opening and closing operation of the fingers slows down, and thus the total motion distance accompanied by opening and closing the fingers also tends to decrease in a later time zone, and the inclination of the straight line also decreases. As the fatigue degree increases, the difference in the inclination between the straight lines also increases. On the other hand, in the case of the healthy person “s”, as illustrated in (b) of FIG. 10, the differences in inclination among the straight lines L8 (y=1.0794x+140.44), L9 (y=1.1723x+181.31), L10 (y=1.2069x+319.4), and L11 (y=1.2232x+250.55) are also small. Note that also in this display mode, each time zone may be displayed in a distinguishable manner by changing not only the line type of the straight line but also its color or the like.

Heretofore, the embodiments of the present invention have been described above with reference to the drawings. However, the present invention is not limited to the above-described embodiments, and can include various modifications. For example, the above-described embodiments have been described in detail in order to describe the present invention in a manner to be easily understandable, and are not necessarily limited to those having all the configurations that have been described. In addition, a part of the configuration in one embodiment can be replaced with the configuration in another embodiment, and the configuration in one embodiment can be added to the configuration in another embodiment. Further, in a part of the configuration in each embodiment, it is possible to add, delete, and replace another configuration.

In addition, some or all of the above-described configurations, functions, processing units, processing means, and the like may be implemented by hardware, for example, by designing them on an integrated circuit. Further, each of the above-described configurations, functions, and the like may be implemented with software, by a processor interpreting and executing a program for achieving each function. Information such as a program, a table, and a file for achieving each function may be stored in a recording apparatus such as a memory, a hard disk, and a solid state drive (SSD), or a recording medium such as an IC card, an SD card, and a DVD, or may be stored in an apparatus on a communication network.

In addition, control lines and information lines that are considered to be necessary for the description are illustrated, and all the control lines and the information lines of a product are not necessarily illustrated. In practice, it may be considered that almost all the configurations are connected with one another.

### REFERENCE SIGNS LIST

2 Tapping sensor
10 Measurement detector
30 Processor
32 Average value data generation circuit
33 Feature amount extraction path
34 Time-series data generation circuit
37 Display

1. A finger tapping measurement processing apparatus comprising:
a measurement detector including a tapping sensor that magnetically detects a finger tapping motion that is an opening and closing motion of two fingers; and a processor that processes measurement data measured by the measurement detector, wherein
the processor includes:
a feature amount extraction circuit that extracts, as quantitative data, a feature amount related to a fatigue degree of the fingers from detection information detected by the tapping sensor; and
a time-series data generation circuit that generates time-series data of the feature amount extracted by the feature amount extraction circuit.

2. The finger tapping measurement processing apparatus according to claim 1, wherein the feature amount extracted by the feature amount extraction circuit includes at least one of a phase difference between tapping waveforms of a right hand and a left hand in the finger tapping motion of cyclically opening and closing the fingers, a total motion distance accompanied by opening and closing the fingers, a tapping cycle in the finger tapping motion, and a maximum separated distance between the two fingers.

3. The finger tapping measurement processing apparatus according to claim 1, wherein the time-series data generation circuit generates graphed time-series data.

4. The finger tapping measurement processing apparatus according to claim 1, further comprising a display that displays the time-series data generated by the time-series data generation circuit.

5. The finger tapping measurement processing apparatus according to claim 4, wherein the processor further includes an average value data generation circuit that generates average value data related to each feature amount of a plurality of subjects whose finger tapping motions are measured by the measurement detector, and the time-series data generation circuit generates display data to be displayed on the display in such a manner that a reference line indicating the average value data is superimposed on the time-series data.

6. The finger tapping measurement processing apparatus according to claim 4, wherein the time-series data generation circuit generates display data to be displayed on the display in such a manner that past history data in the time-series data of an identical feature amount is arranged side by side.

7. The finger tapping measurement processing apparatus according to claim 4, wherein the time-series data generation circuit divides a time axis of the time-series data of the feature amount into a plurality of time zones each having an equal elapsed time, generates each section data that is the time-series data corresponding to each time zone, and also generates display data to be displayed on the display in such a manner that the each section data is arranged side by side along a continuous time series to be distinguishable from each other.

8. The finger tapping measurement processing apparatus according to claim 4, wherein the time-series data generation circuit divides a time axis of the time-series data of the feature amount into a plurality of time zones each having an equal elapsed time, generates each section data that is the time-series data corresponding to each time zone, and also generates display data to be displayed on the display in such a manner that the each section data is arranged side by side in each time series in each time zone to be distinguishable from each other.

9. A finger tapping measurement processing method for measuring a finger tapping motion that is an opening and closing motion of two fingers and processing a measurement result, the finger tapping measurement processing method comprising:
a detection step of magnetically detecting the finger tapping motion:
a feature amount extraction step of extracting, as quantitative data, a feature amount related to a fatigue degree of the fingers from detection information detected in the detection step; and
a time-series data generation step of generating time-series data of the feature amount extracted in the feature amount extraction step.

10. The finger tapping measurement processing method according to claim 9, wherein the feature amount extracted in the feature amount extraction step includes at least one of a phase difference between tapping waveforms of a right hand and a left hand in the finger tapping motion of cyclically opening and closing the fingers, a total motion distance accompanied by opening and closing the fingers, a tapping cycle in the finger tapping motion, and a maximum separated distance between the two fingers.

11. The finger tapping measurement processing method according to claim 9, wherein the time-series data generation step generates graphed time-series data.

12. The finger tapping measurement processing method according to claim 9, further comprising a display step of displaying, on a display, the time-series data generated in the time-series data generation step.

13. The finger tapping measurement processing method according to claim 12, further comprising an average value data generation step of generating average value data related to each feature amount of a plurality of subjects whose finger tapping motions are detected in the detection step, wherein the time-series data generation step generates display data to be displayed on the display in such a manner that a reference line indicating the average value data is superimposed on the time-series data.

14. The finger tapping measurement processing method according to claim 12, wherein the time-series data generation step generates display data to be displayed on the display in such a manner that past history data in the time-series data of an identical feature amount is arranged side by side.

15. The finger tapping measurement processing method according to claim 12, wherein the time-series data generation step divides a time axis of the time-series data of the feature amount into a plurality of time zones each having an equal elapsed time, generates each section data that is the time-series data corresponding to each time zone, and also generates display data to be displayed on the display in such a manner that the each section data is arranged side by side along a continuous time series to be distinguishable from each other.

16. The finger tapping measurement processing method according to claim 12, wherein the time-series data generation step divides a time axis of the time-series data of the feature amount into a plurality of time zones each having an equal elapsed time, generates each section data that is the time-series data corresponding to each time zone, and also generates display data to be displayed on the display in such a manner that the each section data is arranged side by side in each time series in each time zone to be distinguishable from each other.

17. A computer program for processing a measurement result of a finger tapping motion that is an opening and closing motion of two fingers, the computer program causing a computer to perform:
a tapping data acquisition step of acquiring detection data from a tapping sensor that magnetically detects the finger tapping motion:
a feature amount extraction step of extracting, as quantitative data, a feature amount related to a fatigue degree of the fingers from the detection data acquired in the tapping data acquisition step; and
a time-series data generation step of generating time-series data of the feature amount extracted in the feature amount extraction step.

18. The computer program according to claim 17, wherein the feature amount extracted in the feature amount extraction step includes at least one of a phase difference between tapping waveforms of a right hand and a left hand in the finger tapping motion of cyclically opening and closing the fingers, a total motion distance accompanied by opening and closing the fingers, a tapping cycle in the finger tapping motion, and a maximum separated distance between the two fingers.

19. The computer program according to claim 17, wherein the time-series data generation step generates graphed time-series data.

20. The computer program according to claim 17, further causing the computer to process a display step of displaying, on a display, the time-series data generated in the time-series data generation step.

21. The computer program according to claim 20, further causing the computer to process an average value data generation step of generating average value data related to each feature amount of a plurality of subjects whose finger tapping motions are detected in the detection step, wherein the time-series data generation step generates display data to be displayed on the display in such a manner that a reference line indicating the average value data is superimposed on the time-series data.

22. The computer program according to claim 20, wherein the time-series data generation step generates display data to be displayed on the display in such a manner that past history data in the time-series data of an identical feature amount is arranged side by side.

23. The computer program according to claim 20, wherein the time-series data generation step divides a time axis of the time-series data of the feature amount into a plurality of time zones each having an equal elapsed time, generates each section data that is the time-series data corresponding to each time zone, and also generates display data to be displayed on the display in such a manner that the each section data is arranged side by side along a continuous time series to be distinguishable from each other.

24. The computer program according to claim 20, wherein the time-series data generation step divides a time axis of the time-series data of the feature amount into a plurality of time zones each having an equal elapsed time, generates each section data that is the time-series data corresponding to each time zone, and also generates display data to be displayed on the display in such a manner that the each section data is arranged side by side in each time series in each time zone to be distinguishable from each other.
